(19) United States
(12) Patent Application Publication
(10) Pub. No.: US 2014/0107498 A1
(43) Pub. Date: Apr. 17, 2014

(54) Wearable Apparatus and Associated Methods

(71) Applicant: NOKIA CORPORATION, Espoo (FI)

(72) Inventors: Chris Bower, Ely (GB); Marc Bailey, Cambridge (GB); Claudio Marinelli, Highfields Caldecote (GB)

(73) Assignee: Nokia Corporation, Espoo (FI)

(21) Appl. No.: 13/653,574

(22) Filed: Oct. 17, 2012

(51) Int. Cl. A61B 6/00

(52) U.S. Cl. 600/479

(57) ABSTRACT

A wearable apparatus including a waveguide configured to act as a conduit for light emitted from an illumination source to a photodetector via an interaction portion of the waveguide, the interaction portion configured to channel the light out of the waveguide to enable interaction of the light with a wearer's body and back into the waveguide to enable detection of the interacted light by the photodetector.

### TECHNICAL FIELD

The present disclosure relates to the field of health and fitness monitors, associated methods and apparatus, and in particular concerns a wearable apparatus comprising a waveguide for directing light from an illumination source to a photodetector via a wearer's body. Certain disclosed example aspects/embodiments relate to portable electronic devices, in particular, so-called hand-portable electronic devices which may be hand-held in use (although they may be placed in a cradle in use). Such hand-portable electronic devices include so-called Personal Digital Assistants (PDAs) and tablet PCs.

The portable electronic devices/apparatus according to one or more disclosed example aspects/embodiments may provide one or more audio/text/video communication functions (e.g. tele-communication, video-communication, and/or text transmission, Short Message Service (SMS)/Multimedia Message Service (MMS)/emailing functions, interactive/non-interactive viewing functions (e.g. web-browsing, navigation, TV/program viewing functions), music recording/playing functions (e.g. MP3 or other format and/or (FM/AM) radio broadcast recording/playing), downloading/sending of data functions, image capture function (e.g. using a (e.g. in-built) digital camera), and gaming functions.

### BACKGROUND

Optical heart rate monitoring (HRM) provides a suitable solution to monitoring heart rate by wearable sensors. The cost and power requirements of optical HRM systems, as well as their relative simplicity, meet most of the stringent requirements of commercial wearable devices. However, the measurement accuracy achieved by optical HRM systems is well below the desired level, even for non-medical applications.

This limitation is intrinsic in the way optical HRM systems operate. They typically work by irradiating the skin with light generated by visible or infrared light emitting diodes (LEDs), which are usually placed in close contact with the skin. A nearby photodetector, also placed in close contact with the skin, measures the light resulting from reflection, absorption and scattering by the skin. Tracking the variations in light reflection, absorption and scattering allows the measurement of the flow of oxy and deoxy-hemoglobin as well as the expansion of blood vessels, thus enabling oxymetry and pulsometry measurements. Combined or in isolation, these provide a measurement of heart rate and blood circulation.

Optical HRM is adversely affected by variations in the distance between the wearable sensor and the skin, as well as the orientation and shape of the skin surface. Any movement of the portion of the user's body to which the wearable sensor is applied can alter the distance therebetween, i.e. the size of the air gap crossed by light when travelling from the LED to the skin, and from the skin to the photodetector. Movements can also alter the position and orientation of the skin relative to any emitting LEDs and receiving photodetectors. This means that movements severely interfere with the periodic and natural variations in reflections, absorption and scattering caused by blood circulation and heart pulses. A number of measurement artifacts are therefore introduced, which blur the desired oxymetry and pulsometry readings. This, combined with photodetector saturation caused by light from an LED reaching the photodetector without interacting with the body, leads to frequent loss of the detected signal and discontinuous HRM.

The apparatus and methods disclosed herein may or may not address this issue.

The listing or discussion of a prior-published document or any background in this specification should not necessarily be taken as an acknowledgement that the document or background is part of the state of the art or is common general knowledge. One or more aspects/embodiments of the present disclosure may or may not address one or more of the background issues.

### SUMMARY

According to a first aspect, there is provided a wearable apparatus comprising a waveguide configured to act as a conduit for light emitted from an illumination source to a photodetector via an interaction portion of the waveguide, the interaction portion configured to channel the light out of the waveguide to enable interaction of the light with a wearer's body and back into the waveguide to enable detection of the interacted light by the photodetector.

The use of a waveguide allows the photodetector and illumination source to be positioned more remotely than in close contact with the skin without necessarily compromising light coupling.

The term “wearable” may be taken to mean that the apparatus is suitable and/or intended to be worn. A number of different characteristics of the apparatus may render it wearable. For example, the materials forming the apparatus may be soft, smooth, lightweight, breathable, hypoallergenic, flexible and/or stretchable. It will be appreciated that excessive stretching, bending and/or compression may have an effect on the transmission of light through the waveguide so the apparatus should have properties to allowable wearability but minimise light leakage, for example, due to excessive bending/compression. Additionally or alternatively, the shape and configuration of the apparatus, or the fit of the apparatus to the wearer, may render the apparatus wearable.

The term “body” may be taken to mean the whole or part of the wearer's body. For example, the wearable apparatus may be configured to be worn around the wearer's arm, leg, wrist, finger or earlobe to enable interaction of the light with these respective body parts (but is not necessarily limited to these examples).

The waveguide may comprise a single waveguide element. The interaction portion may be configured to channel the light out of and back into the single waveguide element. The waveguide may comprise first and second waveguide elements each comprising a respective interaction portion. The interaction portion of the first waveguide element may be configured to channel the light out of the first waveguide element. The interaction portion of the second waveguide element may be configured to channel the light into the second waveguide element.

The waveguide may be a flexible and/or stretchable waveguide. The term “flexible” may be taken to mean that the waveguide can be reversibly bent about one or more axes by the application of an external force on the waveguide. The term “stretchable” may be taken to mean that one or more of the length, width and thickness of the waveguide can be reversibly increased by the application of an external force on the waveguide.

The interaction portion may be configured to be directly attached to the wearer's body. The interaction portion may be provided on a substrate. The substrate may be configured to be attached to the wearer's body.

The interaction portion may be an end portion of the waveguide. The end portion may be attached to the substrate. The end portion may be shaped to reduce the emission and/or acceptance angles of the waveguide. The interaction portion may be a portion of the waveguide located between the ends of the waveguide.

The waveguide may be formed from, on or within the substrate. The substrate may comprise one or more cavities configured to facilitate channelling of the light. The one or more cavities may be positioned proximal to the interaction portion. The substrate may be formed from an auxetic material configured to preserve the shape of the one or more cavities when the substrate undergoes mechanical deformation.

The substrate may be a flexible and/or stretchable substrate. The term “flexible” may be taken to mean that the substrate can be reversibly bent about one or more axes by the application of an external force on the substrate. The term “stretchable” may be taken to mean that one or more of the length, width and thickness of the substrate can be reversibly increased by the application of an external force on the substrate.

The wearable apparatus may comprise an index matching material between the waveguide and the wearer's body to facilitate channelling of the light. The interaction portion may comprise an optical element configured to facilitate channelling of the light. The optical element may be a reflective element, a refractive element, a diffractive element, a scattering element and/or a secondary waveguide comprising any of the aforementioned optical elements.

One end of the single waveguide element may be (e.g. releasably) attachable to the illumination source to enable receipt of the light by the waveguide. The other end of the single waveguide element may be (e.g. releasably) attachable to the photodetector to enable delivery of the light by the waveguide. One end of the single waveguide element may be (e.g. releasably) attachable to both the illumination source and the photodetector to enable receipt and delivery of the light by the waveguide.

An end of the first waveguide element may be (e.g. releasably) attachable to the illumination source to enable receipt of the light by the waveguide. An end of the second waveguide element may be (e.g. releasably) attachable to the photodetector to enable delivery of the light by the waveguide.

The wearable apparatus may comprise the illumination source and/or photodetector. The wearable apparatus may comprise a plurality of illumination sources, photodetectors and waveguides. Each waveguide may be (e.g. releasably) attachable to a respective illumination source and photodetector. The illumination source may comprise one or more light emitting diodes. The photodetector may comprise one or more of a p-n junction, a photoresistor, a photodiode and a phototransistor. The light may comprise one or more of visible, ultraviolet and infrared light.

The wearable apparatus may be one or more of a garment, a watch, a strap for a watch, a patch, a health monitor, a fitness monitor, a heart rate monitor, an electronic device, a portable electronic device, a portable telecommunications device, and a module for any of the aforementioned articles.

The waveguide may be an optical fibre waveguide or a ridge waveguide.

According to a further aspect, there is provided a method comprising enabling the interaction of light with a wearer's body using a wearable apparatus, the wearable apparatus comprising a waveguide configured to act as a conduit for light emitted from an illumination source to a photodetector via an interaction portion of the waveguide, the interaction portion configured to channel the light out of the waveguide to enable interaction of the light with a wearer's body and back into the waveguide to enable detection of the interacted light by the photodetector.

The steps of any method disclosed herein do not have to be performed in the exact order disclosed, unless explicitly stated or understood by the skilled person.

Corresponding computer programs (which may or may not be recorded on a carrier) for implementing one or more of the methods disclosed are also within the present disclosure and encompassed by one or more of the described example embodiments.

The present disclosure includes one or more corresponding aspects, example embodiments or features in isolation or in various combinations whether or not specifically stated (including claimed) in that combination or in isolation. Corresponding means for performing one or more of the discussed functions are also within the present disclosure.

The above summary is intended to be merely exemplary and non-limiting.

### BRIEF DESCRIPTION OF THE FIGURES

A description is now given, by way of example only, with reference to the accompanying drawings, in which:—

FIG. 1 shows a wearable apparatus attached to a wearer's body;

FIG. 2a shows a wearable apparatus comprising first and second waveguide elements according to one embodiment;

FIG. 2b shows a wearable apparatus comprising first and second waveguide elements according to another embodiment;

FIG. 2c shows a wearable apparatus comprising first and second waveguide elements according to yet another embodiment;

FIG. 2d shows a wearable apparatus comprising a single waveguide element according to one embodiment;

FIG. 2e shows a wearable apparatus comprising a single waveguide element according to another embodiment;

FIG. 2f shows a wearable apparatus comprising a single waveguide element according to yet another embodiment;

FIG. 3 shows a wearable apparatus comprising a substrate for attaching waveguides to a wearer's body;

FIG. 4 shows a plurality of waveguides having different end shapes;

FIG. 5 shows a wearable apparatus comprising a waveguide formed on top of a substrate;

FIG. 6a shows a waveguide comprising a coupling element;

FIG. 6b shows a waveguide comprising a secondary waveguide formed thereon;

FIG. 7 shows a wearable apparatus comprising an illumination source and photodetector;

FIG. 8 shows a method of using a wearable apparatus; and

FIG. 9 shows a computer readable medium comprising a computer program for controlling use of a wearable apparatus.

### DESCRIPTION OF SPECIFIC ASPECTS/EMBODIMENTS

As mentioned in the background section, optical HRM is adversely affected by variations in the distance between the wearable sensor (comprising both the illumination source and the photodetector) and the skin, as well as the orientation and shape of the skin surface. A number of solutions have previously been proposed to address this issue.

One option is to strap the sensor tightly to the wearer's body in order to maintain a constant distance between the sensor and the skin. This, however, causes discomfort to the wearer and prevents prolonged use of the device. Another solution involves monitoring the sensor-skin distance and/or movement of the wearer's body, and using this information to compensate for any measurement artifacts. Disadvantages of such an approach include the need for additional computational power, dedicated logic units and digital-analogue converters, which increase the complexity, cost, size and power consumption of the sensor. In addition, the accuracy of the distance and movement information obtained by current devices is insufficient to enable reliable HRM.

There will now be described a wearable apparatus and associated method which may provide a solution to this problem. It should be noted, however, that the wearable apparatus described herein is not limited solely to the monitoring of heart rate, but may be used to monitor any physiological parameters that can be measured/detected using light.

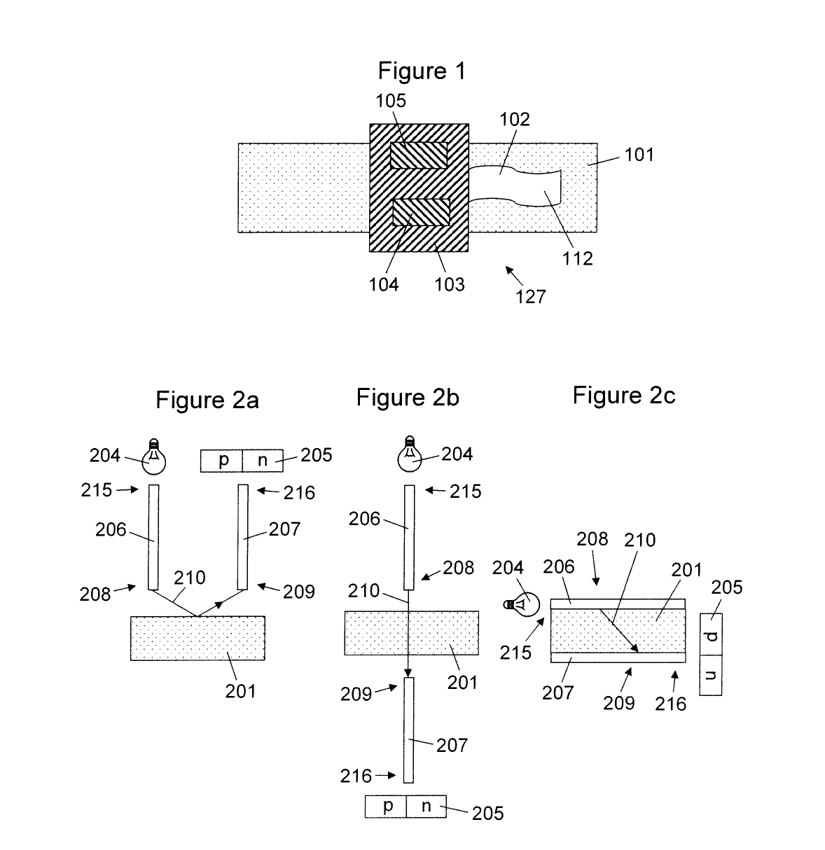

As shown in plan view in FIG. 1, the wearable apparatus 127 comprises a waveguide 102 configured to act as a conduit for light emitted from an illumination source 104 to a photodetector 105 via an interaction portion 112 of the waveguide 102. The interaction portion 112 is a portion of the waveguide 102 which is configured to channel the light out of the waveguide to enable interaction of the light with a wearer's body 101 and back into the waveguide 102 to enable detection of the interacted light by the photodetector 105. The waveguide 102 therefore provides a light path of fixed length between the sensor 103 and the skin 101, and if multiple waveguides were to be deployed, any disruption to the light path caused by local relative movement between the sensor 103 and the user's body 101 would be averaged out and compensated for.

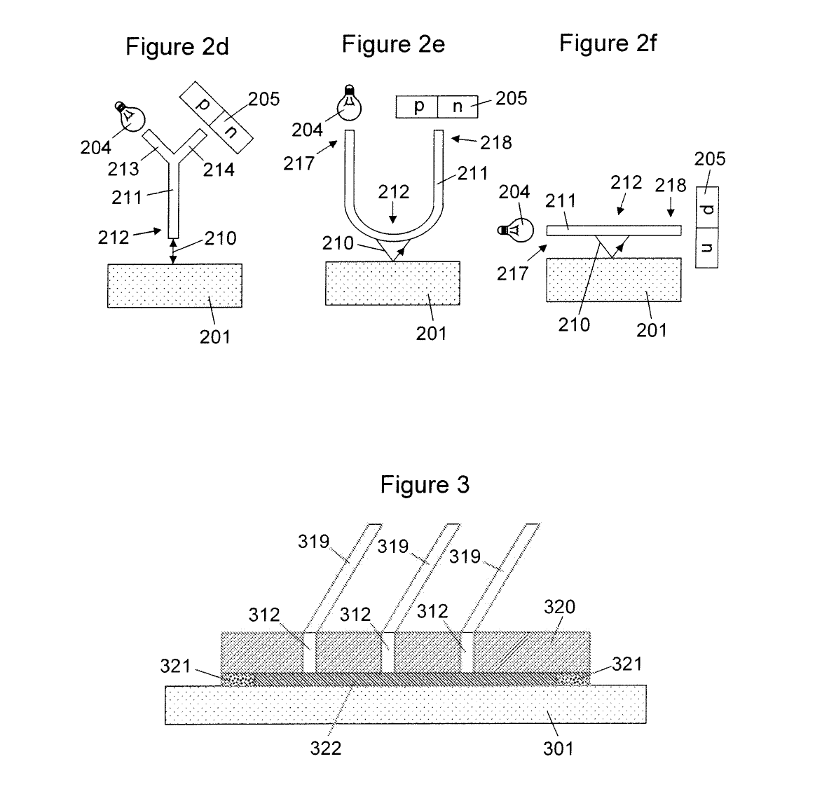

A number of different waveguide configurations are possible, some of which are illustrated schematically in FIGS. 2a to 2f (in side view). As shown, the waveguide may comprise one or more waveguide elements between the sensor and the wearer's body. In some embodiments, the wearable apparatus may be considered to comprise the waveguide/waveguide elements, illumination source and photodetector integrated into a single unit (not shown). In other embodiments, however, the wearable apparatus may comprise the waveguide/waveguide elements per se. In these embodiments, the waveguide/waveguide elements would be configured to be attached to an illumination source and/or photodetector to provide a single unit (not shown). Although not shown in FIGS. 2a to 2f, one or more of the waveguide/waveguide elements, illumination source and photodetector may be provided on a supporting substrate to provide the single unit. Currently available illumination sources and photodetectors used in HRM, for example, could be used.

In FIG. 2a, the waveguide comprises a first waveguide element 206 configured to enable the transfer of light 210 between the illumination source 204 and the wearer's body 201, and a second waveguide element 207 configured to enable the transfer of light 210 between the wearer's body 201 and the photodetector 205. The first waveguide element 206 comprises an interaction portion 208 configured to channel the light 210 out of the first waveguide element 206 towards the wearer's body 201 for interaction therewith, and the second waveguide element 207 comprises an interaction portion 209 configured to channel the light 210 from the wearer's body 201 into the second waveguide element 207 following interaction with the wearer's body 201. In this case, the interaction portions 208, 209 of the first 206 and second 207 waveguide elements are end portions of the respective waveguide elements 206, 207. The configuration of FIG. 2a may be used to detect light 210 which has been reflected or scattered from the wearer's body 201.

FIG. 2b shows a configuration which can be used to detect light 210 which has travelled through the wearer's body 201. To achieve this, the first waveguide element 206 is positioned on one side of the wearer's body 201, and the second waveguide element 207 is positioned on another side (e.g. the opposite side) of the wearer's body 201. As with the previous configuration, the interaction portions 208, 209 of the first 206 and second 207 waveguide elements are end portions of the respective waveguide elements 206, 207. The arrangement of FIG. 2b could be used, for example, to monitor heart rate by measuring the absorption of light 210 by blood in the wearer's finger tip or earlobe (or any other body part 201 through which the light 210 is able to travel).

In the examples shown in FIGS. 2a and 2b, the waveguide elements 206, 207 are configured to be attached (either directly or indirectly, as described later) to the wearer's body 201 end-on. FIG. 2c, however, shows an atternative arrangement in which the first 206 and second 207 waveguide elements are configured to lie substantially parallel to the wearer's body 201. In this example, the interaction portions 208, 209 of the first 206 and second 207 waveguide elements are portions of the respective waveguide elements 206, 207 located between the ends of the waveguide elements 206, 207. The interaction portions 208, 209 are configured such that the light 210 which exits the first waveguide element 206 travels through the wearer's body 201 and is received by the second waveguide element 207.

To allow the transfer of light 210 between the illumination source 204 and the photodetector 205 in FIGS. 2a-2c, an end 215 of the first waveguide element 206 is (e.g. releasably or permanently) attachable to the illumination source 204 to enable receipt of the light 210 by the first waveguide element 206, and an end 216 of the second waveguide element 207 is (e.g. releasably or permanently) attachable to the photodetector 205 to enable delivery of the light 210 by the second waveguide element 207. The ends 215, 216 of the waveguide elements 206, 207 (and parts of the illumination source 204 and photodetector 205) may be configured to enable direct attachment of the waveguide elements 206, 207 to the illumination source 204 and photodetector 205. Alternatively, attachment of the waveguide elements 206, 207 to the illumination source 204 and photodetector 205 may be made indirectly via optical connectors (not shown). Index matching materials (not shown) may also be used between the illumination source 204/photodetector 205 and the waveguide elements 206, 207 to reduce the reflection, refraction, diffraction and/or scattering of any light 210 which impinges upon the interfaces thereof at angles other than normal incidence. This feature therefore helps to reduce optical losses in the light path.

FIG. 2d shows an embodiment comprising a single waveguide element 211 configured to enable both the transfer of light 210 between the illumination source 204 and the wearer's body 201, and the transfer of light 210 between the wearer's body 201 and the photodetector 205. The single waveguide element 211 comprises an interaction portion 212 configured to channel the light 210 out of the single waveguide element 211 towards the wearer's body 201 for interaction therewith, and channel the light 210 from the wearer's body 201 back into the single waveguide element 211 following interaction with the wearer's body 201. In this case, the interaction portion 212 of the single waveguide element 211 is an end portion of the waveguide element 211.

To enable the receipt and delivery of light 210 using a single waveguide element 211, one end of the waveguide element may be attachable to both the illumination source 204 and the photodetector 205. As shown in FIG. 2d, this may be achieved by splitting the end of the single waveguide element 211 into two sections 213, 214, each section 213, 214 optically connected to the body of the single waveguide element 211. One section 213 of the end is attachable to the illumination source 204 and the other section 214 is attachable to the photodetector 205.

This embodiment requires the transmitted and detected light beams to be separated from one another in the single waveguide element 211. In particular, this may be performed by applying periodic, non-overlapping light pulses to allow time for detection between each pulse. Additionally or alternatively, wavelength shifting, frequency shifting or concentric waveguide portions (not shown) for the transmitted and detected light beams may be used.

Rather than splitting an end of the single waveguide element 211, one end 217 of the single waveguide element 211 may be attachable to the illumination source 204 to enable receipt of the light 210 by the single waveguide element 211, and the other end 218 of the single waveguide element 211 may be attachable to the photodetector 205 to enable delivery of the light 210 by the single waveguide element. This configuration is illustrated in FIG. 2e. In this case, the interaction portion 212 used to channel the light 210 between the single waveguide element 211 and the user's body 201 is a portion of the single waveguide element 211 located between the ends 217, 218 of the waveguide element 211.

Another embodiment is shown in FIG. 2f, in which the single waveguide element 211 is configured to lie substantially parallel to the wearer's body 201 to enable the detection of light 210 which has been reflected or scattered from the wearer's body 201. As with the previous embodiment, the interaction portion 212 used to channel the light 210 between the single waveguide element 211 and the user's body 201 is a portion of the single waveguide element 211 located between the ends 217, 218 of the waveguide element 211.

Attachment of the single waveguide element 211 in FIGS. 2d to 2f to the illumination source 204 and photodetector 205 may be made directly or indirectly (as described with reference to FIGS. 2a to 2c). An index matching material (not shown) may also be used to help reduce the optical losses at any interfaces therebetween. The refractive index matching material can be a liquid or a gel, or it may be an elastomeric polymer layer which is deformable to enable better contact with the skin. Whilst any liquid is likely to give an improvement by reducing the step change in going from the refractive index of the light source or fiber, typically n˜1.4 into air having n=1, liquids which are non volatile, and biocompatible are preferred such as heavy paraffin oils, for instance ‘Nujol’ is frequently used in medical applications to couple ultrasound probes to the skin, or halogenated liquids such as Fluorolube can also be applied. Another index matching gel (IMG) is a silicone based synthetic fluid that is combined with insoluble microscopic powders to produce a thixotropic gel. IMG can be purchased as a ready-to-use, single component material requiring no curing. It is highly inert and chemically stable within a temperature range of −59° C. to in excess of 270° C. IMGs can be produced with different specific RIs and can be purchased from suppliers such as Nye Lubricants Inc.

The waveguide may be any type of optical conduit suitable for transferring light from one place to another (e.g. an optical fibre waveguide or a ridge waveguide). To allow movement of the wearer's body during use of the device, however, the waveguide and any supporting substrate (described below) should preferably be made from one or more flexible and/or stretchable materials. The waveguide and supporting structure can be made of polymers and contain flexible waveguide suitable for visible and infrared light transmission. Suitable material could include but not be limited to biocompatible polymers such as PEEK Suitable target substrate 14 may include, but are not necessarily limited to: Polyethylene Terephthalate (PET), Polyethylene Naphthalate (PEN), Polyimide (PI), Polycarbonate (PC), Polyethylene (PE), Polyurethane (PU), Polymethylmethacrylate (PMMA), Polystyrene (PS), natural rubbers such as; Polyisoprenes, Polybutadienes, Poiychloraprenes, Polyisobutylenes, Nitrite Butadienes and Styrene Butadienes, saturated elastomeric materials such as; Polydimethylsiloxane (PDMS), Silicone rubbers, Fluorosilicone rubbers, Fluoroelastomers, Perfluoroelastomers, Ethylene Vinyl Acetate (EVA) Thermoplastic Elastomers such as Styrene Block copolymers, Thermoplastic polyolefins, Thermoplastic vuicanisates, Thermoplastic Polyurethane (TPU) Thermoplastic Copolyesters, Melt processable rubbers.

Optical waveguides typically comprise a core material surrounded by a cladding material of lower refractive. In some cases, the cladding material may simply be the external medium (e.g. air) surrounding the waveguide rather than being part of the waveguide itself. The difference in refractive index causes the light to be confined within the core by total internal reflection. With ridge waveguides, the core forms a ridge on or within a substrate, and the surrounding substrate material serves as (at least part of) the cladding. The core may be created by patterning the substrate (e.g. a polymer substrate) using a laser or hot-embossing technique to vary the refractive index of one or more specific regions. Alternatively, the core (and even the cladding) may be formed on top of the substrate using lithographic processes, or could be made separately from the substrate and subsequently attached thereto. Suitable materials for the core and cladding of the waveguide include Zen Photonics' UV curable resins ZPU120460 (refractive index of 1.47 at 850 nm) and ZPU12-450 (refractive index of 1.46 at 850 nm), respectively. Another option is to form the core and cladding from SU-8 photoresist and Ticona's Topas cycloolefin copolymer (COC), respectively.

At least the interaction portion of the waveguide may be configured for direct or indirect attachment to the wearer's body. Attachment of the waveguide to the wearer's body is important to minimise relative movement therebetween. Direct attachment can be performed using an adhesive (e.g. a hypoallergenic adhesive as used in some medical dressings) between the waveguide and the skin. Alternatively, the interaction portion (or a greater portion of the waveguide) could be provided on a substrate, and the substrate (comprising the waveguide) could be attached to the wearer's body, e.g. using an adhesive. In the latter case, the substrate is used to support and/or form the waveguide, and may be useful when the sensor comprises multiple waveguides or waveguide elements.

FIG. 3 shows three waveguides 319 attached to a supporting substrate 320 which itself is attached to the wearer's body 301 using an adhesive 321. The substrate 320 and waveguides 319 may be considered to form the whole or part of the wearable apparatus. In this example, each waveguide 319 is attached to the substrate by an end portion 312 (i.e. the interaction portion). In addition, an index matching material 322 is located between the substrate 320 and the wearer's body 301 to facilitate channelling of the light.

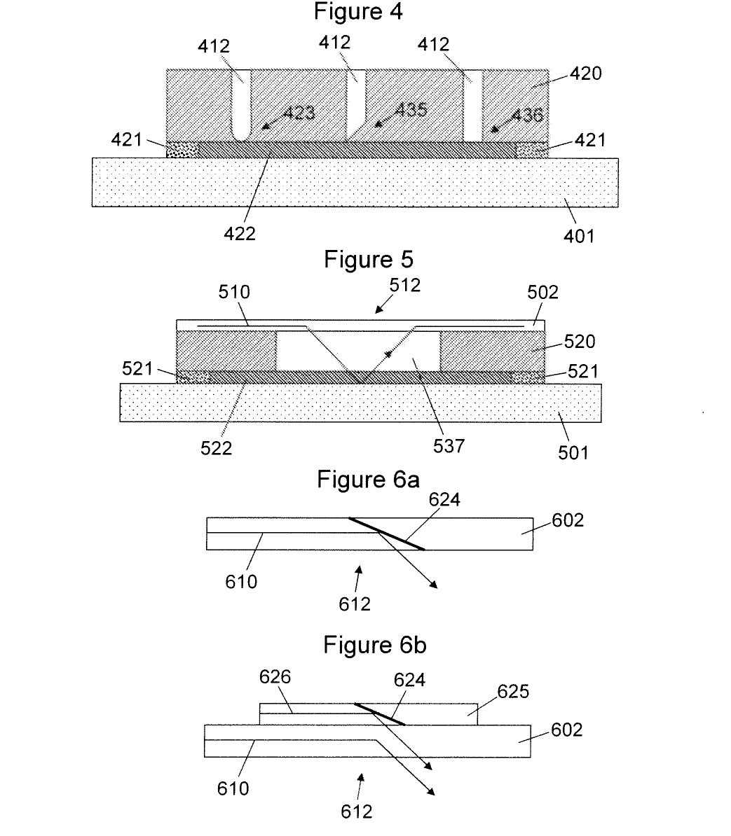

As mentioned previously in the background section, photodetector saturation caused by light from the illumination source reaching the photodetector without interacting with the wearer's body can lead to frequent loss of the detected signal and discontinuous HRM. One way of addressing this issue is to shape the interaction portion(s) of the waveguide to reduce the emission and/or acceptance angles of the waveguide so that only light which has interacted with the wearer's body is transferred by the waveguide to the photodetector. In relation to the example shown in FIG. 3, FIG. 4 shows a few different end shapes that could be used to reduce the emission and/or acceptance angles. These include hemispherical 423, inclined 435 and planar 436 end shapes.

FIG. 5 shows a waveguide 502 formed from, or on top of, a substrate 520 which itself is attached to the wearer's body 501 using an adhesive 521. In this example, the interaction portion 512 is a portion of the waveguide 502 located between the ends of the waveguide 502, and the substrate 520 comprises a cavity 537 to facilitate channelling of the light 510. The substrate 520, waveguide 502 and cavity 537 may be considered to form the whole or part of the wearable apparatus. The cavity 537 may be formed simply by removing substrate material from beneath the waveguide 502, and therefore reduces absorption, reflection or scattering of the light 510 by said substrate material. The cavity 537 may also improve the flexibility and/or stretchability of the substrate 520. In additional, the cavity 537 may be filled with an index matching material 522 to reduce the reflection, refraction, diffraction and/or scattering of the light 510 at the interfaces between the waveguide 501, the substrate 520 and the wearer's body 501. The substrate 520 may be formed from an auxetic material configured to preserve the shape of the cavity 537 when the substrate 520 undergoes mechanical deformation during use of the wearable apparatus.

As described previously, the interaction portion of the waveguide is used to channel the light between the waveguide and the wearer's body. To perform this function, the interaction portion may be bent with respect to the body of the waveguide (as shown in FIG. 3), or it may be shaped in a particular way (as shown in FIG. 4). Additionally or alternatively, the interaction portion may channel light out of and back into the waveguide via direct end-facet emission and collection (respectively), or it may comprise an optical element. The optical element could, for example, be a reflective element, a refractive element, a diffractive element, a scattering element or a secondary waveguide comprising any of the aforementioned optical elements. In FIG. 6a, the interaction portion 612 of the waveguide 602 comprises a reflective element 624 configured to cause the light 610 to exit the waveguide 602.

In FIG. 6b, a similar result is achieved using a secondary waveguide 625 in proximity to the (primary) waveguide 602. In this case, the secondary waveguide 625 is positioned sufficiently close to the primary waveguide 602 that the evanescent field generated by the light wave 610 propagating through the primary waveguide 602 gives rise to an evanescent wave 626 in the secondary waveguide 625 which couples to the light wave 610 in the primary waveguide 602. As a result of the optical coupling between the two light waves 610, 626, an optical element 624 in the secondary waveguide 625 can be used to channel the light wave 610 to and from the primary waveguide 602. The use of a secondary waveguide 625 can help to mitigate power loss associated with reflective and diffractive elements in the primary waveguide 602.

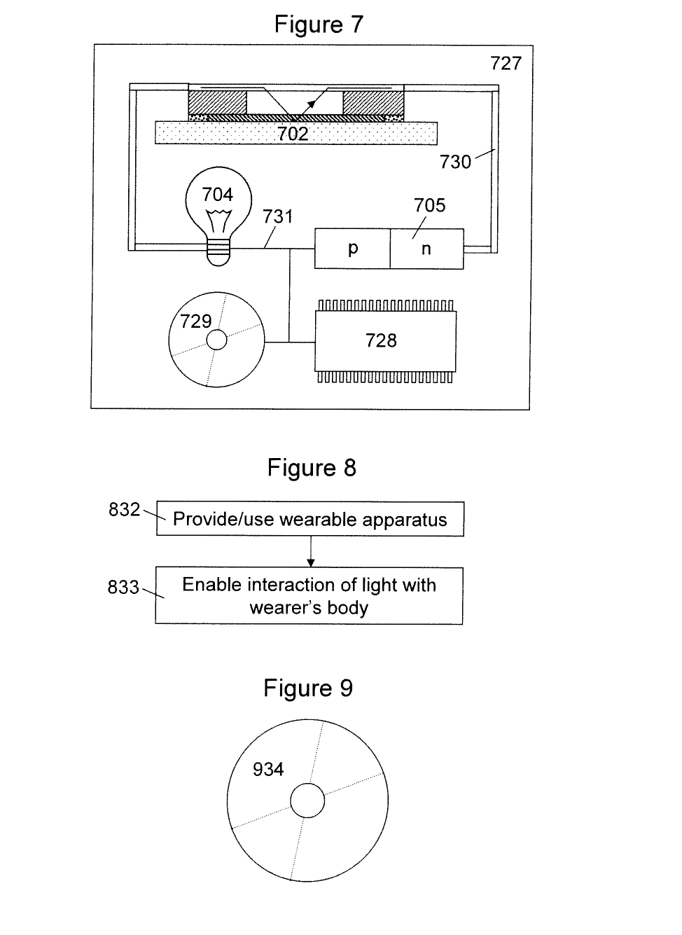

FIG. 7 shows one example of a wearable apparatus 727 comprising the waveguide 702 described herein. The wearable apparatus 727 also comprises the illumination source 704 and photodetector 705 described previously, as well as a processor 728 and a storage medium 729 (although in other examples, this may not necessarily be the case). The illumination source 704 is optically connected to the photodetector 705 by the waveguide 702 (and possibly one or more optical connectors 730). In addition, the illumination source 704, photodetector 705, processor 728 and storage medium 729 are electrically connected to one another by a data bus 731.

The wearable apparatus 727 may be one or more of a garment, a watch, a strap for a watch, a patch, a health monitor, a fitness monitor, a heart rate monitor, an electronic device, a portable electronic device, a portable telecommunications device, and a module for any of the aforementioned devices.

The illumination source 704 is configured to generate light of one or more wavelengths, and the photodetector 705 is configured to detect light generated by the illumination source 704. The waveguide 702 is configured to act as a conduit for light emitted from the illumination source 704 to the photodetector 705 via an interaction portion of the waveguide 702. The interaction portion of the waveguide 702 is configured to channel the light out of and back into the waveguide 702 to enable interaction of the light with a wearer's body.

In some embodiments, the wearable apparatus 727 may comprise a plurality of illumination sources 704, photodetectors 705 and waveguides 702 (e.g. arranged to form an array of sensors). For example, each waveguide 702 may be attachable to a respective illumination source 704 and photodetector 705, or multiple waveguides 702 might be attachable between each illumination source 704 and photodetector 705. An array of sensors could be used to monitor the same physiological parameter at different points on the wearer's body, or to monitor different physiological parameters at the same or different points on the wearer's body.

The processor 728 is configured for general operation of the wearable apparatus 727 by providing signalling to, and receiving signalling from, the other components to manage their operation. The storage medium 729 is configured to store computer code configured to perform, control or enable operation of the wearable apparatus 727. The storage medium 729 may also be configured to store settings for the other components. The processor 728 may access the storage medium 729 to retrieve the component settings in order to manage the operation of the other components.

In addition, the processor 728 may be configured to receive measurements (e.g. voltage, current and/or resistance measurements) from the photodetector 705 and process this data as part of the monitoring process. For example, the processor 728 may be configured to determine the wearer's heart rate based on the voltage, current and/or resistance measurements received from the photodetector 705, and may provide signalling to enable the determined heart rate to be presented to the wearer via an electronic display (which might also form part of the wearable apparatus 727).

Furthermore, the storage medium 729 may be configured to store threshold values (e.g. threshold voltages, currents and/or resistances) indicating the occurrence of a heart beat. The processor 728 may compare the measurements received from the photodetector 705 with the stored threshold values to determine when, and how often, heart beats have occurred.

The processor 728 may be a microprocessor, including an Application Specific Integrated Circuit (ASIC). The storage medium 729 may be a temporary storage medium such as a volatile random access memory. On the other hand, the storage medium 729 may be a permanent storage medium such as a hard disk drive, a flash memory, or a non-volatile random access memory.

The principles of health and/or fitness monitoring using output signals from optical systems are well known in the art and have therefore not been described herein. It will be appreciated, however, that these principles could be used with the present apparatus to enable the monitoring of health and/or fitness.

The main steps 832-833 of a method of using the wearable apparatus 727 are illustrated schematically in FIG. 8.

FIG. 9 illustrates schematically a computer/processor readable medium 934 providing a computer program according to one embodiment. In this example, the computer/processor readable medium 934 is a disc such as a digital versatile disc (DVD) or a compact disc (CD). In other embodiments, the computer/processor readable medium 934 may be any medium that has been programmed in such a way as to carry out an inventive function. The computer/processor readable medium 934 may be a removable memory device such as a memory stick or memory card (SD, mini SD or micro SD).

The computer program may comprise computer code configured to control the interaction of light with a wearer's body using a wearable apparatus, the wearable apparatus comprising a waveguide configured to act as a conduit for light emitted from an illumination source to a photodetector via an interaction portion of the waveguide, the interaction portion configured to channel the light out of the waveguide to enable interaction of the light with a wearer's body and back into the waveguide to enable detection of the interacted light by the photodetector.

Other embodiments depicted in the figures have been provided with reference numerals that correspond to similar features of earlier described embodiments. For example, feature number 1 can also correspond to numbers 101, 201, 301 etc. These numbered features may appear in the figures but may not have been directly referred to within the description of these particular embodiments. These have still been provided in the figures to aid understanding of the further embodiments, particularly in relation to the features of similar earlier described embodiments.

It will be appreciated to the skilled reader that any mentioned apparatus/device and/or other features of particular mentioned apparatus/device may be provided by apparatus arranged such that they become configured to carry out the desired operations only when enabled, e.g. switched on, or the like. In such cases, they may not necessarily have the appropriate software loaded into the active memory in the non-enabled (e.g. switched off state) and only load the appropriate software in the enabled (e.g. on state). The apparatus may comprise hardware circuitry and/or firmware. The apparatus may comprise software loaded onto memory. Such software/computer programs may be recorded on the same memory/processor/functional units and/or on one or more memories/processors/functional units.

In some embodiments, a particular mentioned apparatus/device may be pre-programmed with the appropriate software to carry out desired operations, and wherein the appropriate software can be enabled for use by a user downloading a “key”, for example, to unlock/enable the software and its associated functionality. Advantages associated with such embodiments can include a reduced requirement to download data when further functionality is required for a device, and this can be useful in examples where a device is perceived to have sufficient capacity to store such pre-programmed software for functionality that may not be enabled by a user.

It will be appreciated that any mentioned apparatus/circuitry/elements/processor may have other functions in addition to the mentioned functions, and that these functions may be performed by the same apparatus/circuitry/elements/processor. One or more disclosed aspects may encompass the electronic distribution of associated computer programs and computer programs (which may be source/transport encoded) recorded on an appropriate carrier (e.g. memory, signal).

It will be appreciated that any “computer” described herein can comprise a collection of one or more individual processors/processing elements that may or may not be located on the same circuit board, or the same region/position of a circuit board or even the same device. In some embodiments one or more of any mentioned processors may be distributed over a plurality of devices. The same or different processor/processing elements may perform one or more functions described herein.

It will be appreciated that the term “signalling” may refer to one or more signals transmitted as a series of transmitted and/or received signals. The series of signals may comprise one, two, three, four or even more individual signal components or distinct signals to make up said signalling. Some or all of these individual signals may be transmitted/received simultaneously, in sequence, and/or such that they temporally overlap one another.

With reference to any discussion of any mentioned computer and/or processor and memory (e.g. including ROM, CD-ROM etc), these may comprise a computer processor, Application Specific Integrated Circuit (ASIC), field-programmable gate array (FPGA), and/or other hardware components that have been programmed in such a way to carry out the inventive function.

The applicant hereby discloses in isolation each individual feature described herein and any combination of two or more such features, to the extent that such features or combinations are capable of being carried out based on the present specification as a whole, in the light of the common general knowledge of a person skilled in the art, irrespective of whether such features or combinations of features solve any problems disclosed herein, and without limitation to the scope of the claims. The applicant indicates that the disclosed aspects/embodiments may consist of any such individual feature or combination of features. In view of the foregoing description it will be evident to a person skilled in the art that various modifications may be made within the scope of the disclosure.

While there have been shown and described and pointed out fundamental novel features as applied to different embodiments thereof, it will be understood that various omissions and substitutions and changes in the form and details of the devices and methods described may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment may be incorporated in any other disclosed or described or suggested form or embodiment as a general matter of design choice. Furthermore, in the claims means-plus-function clauses are intended to cover the structures described herein as performing the recited function and not only structural equivalents, but also equivalent structures. Thus although a nail and a screw may not be structural equivalents in that a nail employs a cylindrical surface to secure wooden parts together, whereas a screw employs a helical surface, in the environment of fastening wooden parts, a nail and a screw may be equivalent structures.

1. A wearable apparatus comprising a waveguide configured to act as a conduit for light emitted from an illumination source to a photodetector via an interaction portion of the waveguide, the interaction portion configured to channel the light out of the waveguide to enable interaction of the light with a wearer's body and back into the waveguide to enable detection of the interacted light by the photodetector.

2. The wearable apparatus of claim 1, wherein the waveguide comprises a single waveguide element, and wherein the interaction portion is configured to channel the light out of and back into the single waveguide element.

3. The wearable apparatus of claim 1, wherein the waveguide comprises first and second waveguide elements each comprising a respective interaction portion, and wherein the interaction portion of the first waveguide element is configured to channel the light out of the first waveguide element, and the interaction portion of the second waveguide element is configured to channel the light into the second waveguide element.

4. The wearable apparatus of claim 1, wherein the waveguide is a flexible and/or stretchable waveguide.

5. The wearable apparatus of claim 1, wherein the interaction portion is provided on a substrate, the substrate configured to be attached to the wearer's body.

6. The wearable apparatus of claim 5, wherein the interaction portion is an end portion of the waveguide, and wherein the end portion is attached to the substrate.

7. The wearable apparatus of claim 6, wherein the end portion is shaped to reduce the emission and/or acceptance angles of the waveguide.

8. The wearable apparatus of claim 1, wherein the interaction portion is a portion of the waveguide located between the ends of the waveguide.

9. The wearable apparatus of claim 5, wherein the waveguide is formed from, on or within the substrate.

10. The wearable apparatus of claim 5, wherein the substrate comprises one or more cavities configured to facilitate channelling of the light, the one or more cavities positioned proximal to the interaction portion.

11. The wearable apparatus of claim 10, wherein the substrate is formed from an auxetic material configured to preserve the shape of the one or more cavities when the substrate undergoes mechanical deformation.

12. The wearable apparatus of claim 5, wherein the substrate is a flexible and/or stretchable substrate.

13. The wearable apparatus of claim 5, wherein the wearable apparatus comprises an index matching material between the waveguide and the wearer's body to facilitate channelling of the light.

14. The wearable apparatus of claim 1, wherein the interaction portion comprises an optical element configured to facilitate channelling of the light, and wherein the optical element is a reflective element, a refractive element, a diffractive element, a scattering element and/or a secondary waveguide comprising any of the aforementioned optical elements.

15. The wearable apparatus of claim 1, wherein the wearable apparatus comprises the illumination source and/or photodetector.

16. The wearable apparatus of claim 1, wherein the wearable apparatus comprises a plurality of illumination sources, photodetectors and waveguides, and wherein each waveguide is attachable to a respective illumination source and photodetector.

17. The wearable apparatus of claim 1, wherein the wearable apparatus is one or more of a garment, a watch, a strap for a watch, a patch, a health monitor, a fitness monitor, a heart rate monitor, an electronic device, a portable electronic device, a portable telecommunications device, and a module for any of the aforementioned articles.

18. The wearable apparatus of claim 1, wherein the waveguide is an optical fibre waveguide or a ridge waveguide.

19. A method comprising enabling the interaction of light with a wearer's body using a wearable apparatus, the wearable apparatus comprising a waveguide configured to act as a conduit for light emitted from an illumination source to a photodetector via an interaction portion of the waveguide, the interaction portion configured to channel the light out of the waveguide to enable interaction of the light with a wearer's body and back into the waveguide to enable detection of the interacted light by the photodetector.

20. A computer program, recorded on a carrier, the computer program comprising computer code configured to control the method of claim 19.
